(19) United States
(12) Patent Application Publication
(10) Pub. No.: US 2022/0194912 A1
(43) Pub. Date: Jun. 23, 2022

(54) ELASTOMER GEL FROM EPOXIDIZED VEGETABLE OIL AND USES THEREOF

(71) Applicant: Momentive Performance Materials Inc., Waterford, NY (US)

(72) Inventors: Xu QIN, Waterford, NY (US); Benjamin FALK, Yorktown Heights, NY (US); Mana TAMAMI, Waterford, NY (US); Yuriy GALABURA, Waterford, NY (US); Amar PAWAR, Waterford, NY (US); Roland WAGNER, Bonn (DE)

(21) Appl. No.: 17/560,026

(22) Filed: Dec. 22, 2021

Related U.S. Application Data
(60) Provisional application No. 63/130,271, filed on Dec. 23, 2020.

(51) Int. Cl. C07D 303/04; A61Q 1/02; A61Q 1/06; A61Q 19/00; A61Q 17/04; C07C 55/02; C07D 305/08; C07C 211/09; C07C 59/01; C07C 31/20; A61K 8/86; C08G 59/32; C08G 59/42; C08G 59/50; C08G 59/62

(52) U.S. Cl. CPC C07D 303/04; C08G 59/625; A61Q 1/06; A61Q 19/007; A61Q 17/04; C07C 55/02; C07D 305/08; C07C 211/09; C07C 59/01; C07C 31/20; A61K 8/86; C08G 59/32; C08G 59/42; C08G 59/4215; C08G 59/5013; A61Q 1/02

(57) ABSTRACT

The present disclosure relates to gel compositions comprising the reaction product of at least one epoxidized molecule and at least one crosslinker, which has the benefit of compatibility with personal care components and the resulting personal care formulations. The present disclosure also relates to methods of preparing gel compositions.

### FIELD

The present disclosure relates to elastomers comprising the reaction product of at least one epoxidized molecule and at least one crosslinker and methods of preparing such compositions. Further, these elastomers can be converted to elastomer gels. The present disclosure also relates to personal care formulations containing such elastomers or elastomer gels.

### BACKGROUND

The personal care industry thrives on being able to deliver multiple performance products based on mixtures of several components, with each having performance characteristics important to or desirable in the final formulation. Silicone gels are commonly added in a variety of personal care formulations to enhance their aesthetics with respect to sensory, texture, rheology, and optical performance. See for example, U.S. Pat. Nos. 4,987,169; 5,654,362; 5,760,116; 6,423,322; and 5,811,487.

However, traditional silicone gels have limited versatility in terms of compatibility with many natural oils, ester oils, and other biobased ingredients which have growing interest in personal care formulation. Moreover, silicone polymers are difficult to degrade, biologically or otherwise. Particular silicone compounds used in various personal care products, cyclic siloxanes D4 (octamethylcyclotetrasiloxane) and D5 (decamethylcyclopentasiloxane), are air and water pollutants and show negative health effects on test animals. In light of the growing environmental awareness, increasing societal concern and new environmental rules and regulations, biodegradable natural polymers gel made from biorenewable raw material with no health and environmental concerns is highly desirable.

Vegetable oils or natural oils, such as soybean oil and linseed oil, are one of the most promising raw materials for the synthesis of renewable compounds. These natural materials are inexpensive, highly abundant, come from reliable and sustainable sources, and have high potential for modification. Vegetable oils are triglycerides which contains different kinds of unsaturated fatty acids and saturated fatty acids with varying carbon chain lengths. Soybean oil, as an example, has three unsaturated fatty acids with average unsaturation degree of 4.6. Epoxidized vegetable oil (EVO) are functionalized triglyceride with oxirane ring moieties. Epoxidized soybean oil (ESO) has been used as a composite (Thielemans W., et al., Journal of Applied Polymer Science, 2002, 83:323-331 and Lu J., et al., Polymer, 2005, 46:71-80), a lubricant, a plasticizer, a thermal stabilizer (Lathi P. S., Applied Catalysis B. Environmental, 2007, 69:207-212, Demertzis P. G. et al., European Polymer Journal, 1991, 27(3):231-235 and Liu P. et al., Polymer Degradation and Stability, 2007, 92:503-508), and flooring materials (U.S. Pat. No. 7,196,124).

Despite its broad usage mentioned above, epoxidized vegetable oil and their derivatives, including polymers, have rarely been used in personal care application. Given these known properties of epoxidized vegetable oil, particularity epoxidized soybean oil, these oils are excellent candidates as a renewable resource monomer units to develop a biodegradable natural polymer gel for personal care formulations.

### BRIEF SUMMARY OF THE DISCLOSURE

In an aspect, the disclosure relates to elastomer comprising the reaction product of at least one epoxidized molecule and at least one crosslinker.

In an aspect, the at least one epoxidized molecule is a compound of formula (I)

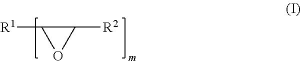

wherein

R¹ is C₆-C_{100,000} alkyl group, C₆-C_{100,000} heteroalkyl group, C₆-C_{100,000} alkene group, C₆-C_{100,000} heteroalkene group, C₆-C_{100,000} alkyne group, C₆-C_{100,000} heteroalkyne group, C₆-C_{100,000} cyclic group, or C₆-C_{100,000} heterocyclic group;

R² is hydrogen, C₁-C_{100,000} alkyl group, C₁-C_{100,000} heteroalkyl group, C₂-C_{100,000} alkene group, C₂-C_{100,000} heteroalkene group, C₂-C_{100,000} alkyne group, C₂-C_{100,000} heteroalkyne group, C₃-C_{100,000} cyclic group, or C₃-C_{100,000} heterocyclic group; and

m is an integer from 2 to 1,000.

In another aspect, the disclosure relates to an elastomer prepared by reacting at least one epoxidized molecule and at least one crosslinker in the presence of a first solvent thereby forming a crosslinking polymer structure.

In yet another aspect, the disclosure relates to a method of preparing an elastomer comprising reacting at least one epoxidized molecule and at least one crosslinker in the presence of a first solvent thereby forming a crosslinking polymer structure.

In another aspect, the disclosure relates to a personal care formulation comprising a gel prepared from an elastomer composition that is the reaction product of at least one epoxidized molecule and at least one crosslinker.

In still yet another aspect, the disclosure relates to use of a gel described herein for personal care formulations.

### DETAILED DESCRIPTION

### Definitions

Unless otherwise indicated, any atom with unsatisfied valences is assumed to have hydrogen atoms sufficient to satisfy the valences.

The singular forms “a,” “an,” and “the” include plural referents unless the context dictates otherwise.

Furthermore, “and/or”, where used herein, is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

As used herein, the following definitions shall apply unless otherwise indicated. For purposes of the present disclosure, the chemical elements are identified in accordance with the Periodic Table of the Elements, CAS version, and the Handbook of Chemistry and Physics, 75^{th} Ed. 1994. Additionally, general principles of organic chemistry are described in “Organic Chemistry,” Thomas Sorrell, University Science Books, Sausalito: 1999, and “March's Advanced Organic Chemistry,” 6^{th}Ed., Smith, M. B. and March, J., eds. John Wiley & Sons, New York: 2007, the entire contents of which are hereby incorporated by reference.

The term “alkyl”, as used herein by itself or as part of a group, refers to a straight- or branched-chain aliphatic hydrocarbon containing one to two hundred carbon atoms, i.e., a C₂-C₂₀₀ alkyl, or the number of carbon atoms designated, e.g., a C₁ alkyl such as methyl, a C₂ alkyl such as ethyl, etc. In one embodiment, the alkyl is a C₂-C₂₀₀ alkyl group. In another embodiment, the alkyl is a C₆-C₆₀ alkyl group. In another embodiment, the alkyl is a C₅-C₂₂ alkyl group. Examples of alkyl group include butyl, octyl, decyl, lauryl, cetyl (palmityl), and stearyl.

The term “alkene”, as used herein by itself or as part of a group, refers to an alkyl group containing one, two, three, or more carbon-to-carbon double bonds. In one embodiment, the alkene group is a C₂-C₂₀₀ alkene group. In another embodiment, the alkene group is a C₆-C₆₀ alkene group. In another embodiment, the alkene group is a C₆-C₆₀ alkene group. In another embodiment, the alkene group is a C₅-C₂₂ alkene group. Examples of alkene include linoleyl, oleyl, dilinoleyl, and trilinoleyl.

The term “alkyne”, as used herein by itself or as part of a group, refers to an alkyl group containing one, two, three, or more carbon-to-carbon triple bonds. In another embodiment, the alkyne is a C₂-C₂₀₀ alkyne group.

The term “cyclic”, as used herein by itself or as part of a group, refers to a stable cyclic compound containing three or more carbon atoms. In an embodiment, the cyclic is a C₃-C₂₀₀ cyclic group. In an embodiment, the cyclic is a C₆-C₆₀ cyclic group. In an embodiment, the cyclic is a C₅-C₂₂ cyclic group. Examples of cyclic compound include benzene, cyclopentane, and cyclohexane.

The term “heteroalkyl”, as used herein by itself or as part of a group, refers to a stable straight or branched chain alkyl radical containing 2 to 200 carbon atoms and at least one heteroatoms, which can be the same or different, selected from O, N, or S, wherein the sulfur atom(s) can optionally be oxidized. The heteroatoms can be placed at any interior position of the heteroalkyl group or at a position at which the heteroalkyl group is attached to the remainder of the molecule. In an embodiment, the heteroalkyl is a C₂-C₆₀ heteroalkyl group. Examples include, but are not limited to, hydroxy ethylene, hydroxy stearyl, glyceryl stearyl, succinyl, adipoyl, and sebacoyl.

The term “heteroalkene”, as used herein by itself or as part of a group, refers to a stable straight or branched chain alkene radical containing 2 to 200 carbon atoms and at least one heteroatoms, which can be the same or different, selected from O, N, or S, wherein the sulfur atom(s) can optionally be oxidized. The heteroatoms can be placed at any interior position of the heteroalkyl group or at a position at which the heteroalkyl group is attached to the remainder of the molecule. Examples of heteroalkene compound include oleoyl, ricinolyl, and linoleoyl

The term “heteroalkyne”, as used herein by itself or as part of a group, refers to a stable straight or branched chain alkyne radical containing 2 to 200 carbon atoms and at least one heteroatoms, which can be the same or different, selected from O, N, or S, wherein the sulfur atom(s) can optionally be oxidized. The heteroatoms can be placed at any interior position of the heteroalkyl group or at a position at which the heteroalkyl group is attached to the remainder of the molecule.

The term “heterocyclic”, as used herein by itself or as part of a group, refers to a stable cyclic compound containing two or more carbon atoms and at least one heteroatom, which can be the same or different, selected from O, N, or S. In an embodiment, the heterocyclic is a C₂-C₂₀₀ heterocyclic group. In an embodiment, the heterocyclic is C₆-C₆₀ heterocyclic group. Examples include, but are not limited to, furan, oxolane, and thiophene.

Various aspect of the disclosure are described in greater detail below.

### Compositions

### A. Elastomer

In an aspect, the present disclosure is directed to elastomer comprising the reaction product of at least one epoxidized molecule and at least one crosslinker.

a. Components

i. Epoxidized Molecule

In an aspect, the at least one epoxidized molecule is a compound of formula (I)

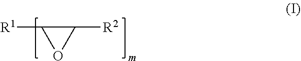

wherein

R¹ is C₆-C_{100,000} alkyl group, C₆-C_{100,000} heteroalkyl group, C₆-C_{100,000} alkene group, C₆-C_{100,000} heteroalkene group, C₆-C_{100,000} alkyne group, C₆-C_{100,000} heteroalkyne group, C₆-C_{100,000} cyclic group, or C₆-C_{100,000} heterocyclic group;

R² is hydrogen, C₁-C_{100,000} alkyl group, C₁-C_{100,000} heteroalkyl group, C₂-C_{100,000} alkene group, C₂-C_{100,000} heteroalkene group, C₂-C_{100,000} alkyne group, C₂-C_{100,000} heteroalkyne group, C₃-C_{100,000} cyclic group, or C₃-C_{100,000} heterocyclic group; and

m is an integer from 2 to 1,000.

In an aspect, the at least one epoxidized molecule may be an epoxidized vegetable oil. In a further aspect, the epoxidized vegetable oil may be selected from the group consisting of epoxidized soybean oil, epoxidized safflower oil, epoxidized linseed oil, epoxidized corn oil, epoxidized sunflower oil, epoxidized olive oil, epoxidized canola oil, epoxidized sesame oil, epoxidized cottonseed oil, epoxidized palm oil, epoxidized rapeseed oil, epoxidized tung oil, epoxidized fish oil, epoxidized peanut oil, epoxidized cuphea oil, epoxidized milkweed oil, epoxidized salicornia oil, epoxidized high oleic safflower oil, epoxidized high oleic soybean oil, epoxidized high oleic peanut oil, epoxidized high oleic sunflower oil, epoxidized high erucic rapeseed oil (crambe oil), and combinations thereof. In a further aspect, the epoxidized vegetable oil may be epoxidized soybean oil, epoxidized linseed oil, and combinations thereof. In yet a further aspect, the epoxidized vegetable oil may be epoxidized soybean oil.

In an aspect, the epoxidized vegetable oil may be prepared by treating a vegetable oil with a peroxyacid to fully epoxidized the double bonds in the vegetable oil. Epoxidation of vegetable oils may be carried out as described by Qureshi et al. Polymer Science and Technology, Vol. 17, Plenum Press, p. 250, the entire contents of which is hereby incorporated by reference. Additional methods for epoxidation of vegetable oils are generally known in the art. In some aspects, the epoxidation reaction is carried to completion. In a non-limiting aspect, the epoxidation may be carried out by reacting milkweed oil with formic acid and hydrogen peroxide at about 75° C.

In some aspects, the degree of epoxidation may be greater than 3 oxirane rings per triglyceride molecule. In other aspects, the degree of epoxidation may be from 3 to 5 oxirane rings per triglyceride molecule. In a further aspect, the degree of epoxidation may be 4 oxirane rings per triglyceride molecule.

ii. Crosslinker

In an aspect, the at least one crosslinker is selected from the group consisting of:

(i) a carboxylic acid comprising the structure of formula (II)

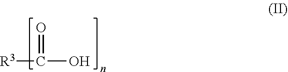

wherein

R³ is C₂-C₂₀₀ alkyl group, C₂-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group; and

n is an integer from 2 to 10;

(ii) an anhydride comprising the structure of formula (III)

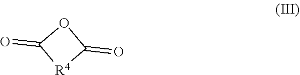

wherein
R⁴ is C₂-C₂₀₀ alkyl group, C₂-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group;

(iii) an amine comprising the structure of formula (IV)

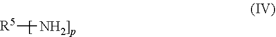

wherein
R⁵ is C₂-C₂₀₀ alkyl group, C₂-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group; and
p is an integer from 2 to 10;

(iv) an alcohol comprising the structure of formula (V)

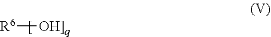

wherein
R⁶ is C₂-C₂₀₀ alkyl group, C₂-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group; and q is an integer from 2 to 10;

(v) a hydroxyl carboxylic acid comprising the structure of formula (VI)

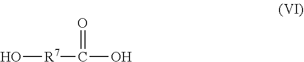

wherein
R⁷ is C₁-C₂₀₀ alkyl group, C₁-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group;

(vi) a amine carboxylic acid comprising the structure of formula (VII)

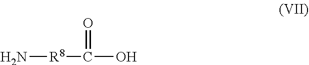

wherein
R⁸ is C₁-C₂₀₀ alkyl group, C₁-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group;
and combinations thereof.

In an aspect, the carboxylic acid comprises the structure of formula (II), wherein R² is C₂-C₆₀ alkyl group, C₂-C₆₀ heteroalkyl group, C₂-C₆₀ alkene group, or C₂-C₆₀ heteroalkene group; and n is an integer from 2 to 10. In a further aspect, R² may be succinyl, adipoyl, sebacoyl, dilinoleyl, or trilinoleyl.

In an aspect, the carboxylic acid comprises the structure of formula (II), wherein n is 2, 3, 4, 5, 6, 7, 8, 9, or 10. In an aspect, the carboxylic acid comprises the structure of formula (II), wherein n is an integer of 2 to 6. In an aspect, the carboxylic acid comprises the structure of formula (II), wherein n is 2, 3, 4, 5, or 6.

In an aspect, the carboxylic acid may be selected from the group consisting of citric acid, isocitric acid, aconitic acid, propane-1,2,3-tricarboxylic acid, trimesic acid, carballylic acid, C₅₄ trimer acid, mellitic acid, reaction product of ricinoleic acid and sebacic acid, reaction product of C₃₆ dimer acid, and C₃₆ dimer diol, and combinations thereof. In a further aspect, the carboxylic acid may be selected from the group consisting of citric acid, C₅₄ trimer acid, and combinations thereof.

In an aspect, the carboxylic acid is a dicarboxylic acid. In an aspect, the dicarboxylic acid may be selected from the group consisting of malonic acid, succinic acid, fumaric acid, glutaric acid, adipic acid, pimelic acid, suberic acid, azelaic acid, sebacic acid, undecanedioic acid, dodecanedioic acid, tridecanedioic acid, hexadecanedioic acid, C₂₁ dimer acid, C₃₆ dimer acid, hydrogenated C₃₆ dimer acid, aspartic acid, glutamic acid, tartaric acid, malic acid, and combinations thereof. In a further aspect, the dicarboxylic acid may be selected from the group consisting of malonic acid, succinic acid, adipic acid, azelaic acid, sebacic acid, undecanedioic acid, dodecanedioic acid, C₂₁ dimer acid, C₃₆ dimer acid, hydrogenated C₃₆ dimer acid, and combinations thereof. In one aspect, the dicarboxylic acid is C₃₆ dimer acid.

In an aspect, the anhydride comprises the structure of formula (III), wherein R⁴ is C₂-C₁₀ alkyl group or C₂-C₁₀ alkene group. In a further aspect, R⁴ is C₂-C₈ alkyl group.

In an aspect, the anhydride may be selected from the group consisting of malonic anhydride, succinic anhydride, maleic anhydride, phthalic anhydride, glutaric anhydride, adipic anhydride, pimelic anhydride, suberic anhydride, azelaic anhydride, sebacic anhydride, undecanedioic anhydride, dodecanedioic anhydride, tridecanedioic anhydride, hexadecanedioic anhydride, C₂₁ dimer acid anhydride, C₃₆ dimer acid anhydride, hydrogenated C₃₆ dimer acid anhydride, aspartic anhydride, glutamic anhydride, tartaric anhydride, malic anhydride, and combinations thereof. In a further aspect, the anhydride may be selected from the group consisting of succinic anhydride, adipic anhydride, sebacic anhydride, C₃₆ dimer acid anhydride, hydrogenated C₃₆ dimer acid anhydride, and combinations thereof.

In an aspect, the amine comprises the structure of formula (IV), wherein p is 2, 3, 4, 5, 6, 7, 8, 9, or 10. In an aspect, the amine comprises the structure of formula (IV), wherein p is an integer of 2 to 6. In an aspect, the amine comprises the structure of formula (IV), wherein p is 2, 3, 4, 5, or 6.

In an aspect, the amine may be selected from the group consisting of 1,2-diaminoethane, propane-1,3-diamine, butane-1,4-diamine, pentane-7,5-diamine, hexane-1,6-diamine, 1,7-heptanediamine, C₃₆ dimer diamine, hydrogenated C₃₆ dimer diamine, C₅₄ trimer triamine, and combinations thereof. In a further aspect, the amine may be selected from the group consisting of hexane-1,6-diamine, C₃₆ dimer diamine, hydrogenated C₃₆ dimer diamine, and combinations thereof.

In an aspect, the amine may be selected from the group consisting of include ethylenediamine, 1,3-diaminopropane (1,3-propanediamine), putrescine (1,4-butanediamine), cadaverine (1,5-pentanediamine), hexamethylenediamine (1,6-hexanediamine), trimethyl-1,6-hexanediamine, 1,7-heptanediamine, 1,8-octanediamine, 1,9-nonanediamine, 1,10-decanediamine, 1,11-undecanediamine, 1,12-dodecanediamine, diphenylethylenediamine, 1,2-diaminopropane, 1,2-diaminocyclohexane, triethylenetetramine, xylylenediamine, phenylenediamine, spermidine, spermine, polyethylenimine, C₃₆ dimer diamine, hydrogenated C₃₆ dimer diamine, poly(ethylene glycol) bis(amine), lysine, and combinations thereof. In some aspects, the amine may be selected from the group consisting of spermidine, spermine, polyethylenimine, and combinations thereof. In other aspects, the amine may be selected from the group consisting of 1,5-pentanediamine, 1,6-hexanediamine, 1,8-octanediamine, 1,10-decanediamine, 1,12-dodecanediamine, C₃₆ dimer diamine, hydrogenated C₃₆ dimer diamine, and combinations thereof.

In an aspect, wherein the amine may be a diamine. In an aspect, the diamine may be selected from the group consisting of ethylenediamine, 1,3-diaminopropane (1,3-propanediamine), putrescine (1,4-butanediamine), cadaverine (1,5-pentanediamine), hexamethylenediamine (1,6-hexanediamine), trimethyl-1,6-hexanediamine, 1,7-heptanediamine, 1,8-octanediamine, 1,9-nonanediamine, 1,10-decanediamine, 1,11-undecanediamine, 1,12-dodecanediamine, diphenylethylenediamine, 1,2-diaminopropane, 1,2-diaminocyclohexane, xylylenediamine, phenylenediamine, polyethylenimine, C₃₆ dimer diamine, hydrogenated C₃₆ dimer diamine, poly(ethylene glycol) bis(amine), lysine, and combinations thereof. In a further aspect, the diamine may be selected from the group consisting of 1,5-pentanediamine, 1,6-hexanediamine, 1,8-octanediamine, 1,10-decanediamine, 1,12-dodecanediamine, C₃₆ dimer diamine, hydrogenated C₃₆ dimer diamine, and combinations thereof.

In an aspect, the alcohol comprises the structure of formula (V), wherein R⁶ is C₂-C₂₀₀ alkyl group, C₂-C₂₀₀ heteroalkyl group, C₂-C₂₀₀ alkene group, or C₂-C₂₀₀ heteroalkene group; and q is an integer from 2 to 10. In a further aspect, the alcohol comprises the structure of formula (V), wherein R⁶ is C₂-C₆₀ alkyl group, C₂-C₆₀ heteroalkyl group, C₂-C₆₀ alkene group, or C₂-C₆₀ heteroalkene group; and q is an integer from 2 to 10.

In an aspect, the alcohol comprises the structure of formula (V), wherein q is 2, 3, 4, 5, 6, 7, 8, 9, or 10. In an aspect, the alcohol comprises the structure of formula (V), wherein q is an integer of 2 to 6. In an aspect, the alcohol comprises the structure of formula (V), wherein q is 2, 3, 4, 5, or 6.

In an aspect, the alcohol may be selected from the group consisting of glycerol, diglycerol, polyglycerol, sorbitan, castor oil, hydrogenated castor oil, sugar alcohol, monosaccharide, disaccharides, oligosaccharide, polysaccharides, tannin, gallic acid, gluconic acid, lactobionic acid, gluconolactone, and combinations thereof. In a further aspect, the alcohol may be selected from the group consisting of glycerol, diglycerol, polyglycerol, castor oil, hydrogenated castor oil, sorbitol, gallic acid, and combinations thereof.

In an aspect, the alcohol may be a diol. In some aspects, the diol may be selected from the group consisting of ethyleneglycol, 1,2-propanediol, 1,3-propanediol, 1,3-butanediol, 1,4-butanediol, 1,2-pentanediol, 1,3-pentanediol, 1,4-pentanediol 1,1,5-pentanediol, 1,2-hexanediol, 1,5-hexanediol, 1,6-hexanediol, C₃₆ dimer diol, hydrogenated C₃₆ dimer diol, and combinations thereof. In a further aspect, the diol may be selected from the group consisting of 1,3-propanediol, 1,3-butanediol, 1,4-butanediol, 1,5-pentanediol, 1,6-hexanediol, C₃₆ dimer diol, hydrogenated C₃₆ dimer diol, and combinations thereof. In one aspect, the alcohol is C₃₆ dimer diol.

In an aspect, the hydroxyl carboxylic acid may be selected from the group consisting of glycolic acid, lactic acid, salicylic acid, tropic acid, ricinoleic acid, isoricinoleic acid, lesquerolic acid, densipolic acid, auricolic acid, dimorphecolic acid, hydroxy palmitoleic acid, hydroxy palmitic acid, hydroxy oleic acid, 2-hydroxy stearic acid, 12-hydroxy stearic acid, and combinations thereof. In a further aspect, the hydroxyl carboxylic acid may be selected from the group consisting of ricinoleic acid, 12-hydroxy stearic acid, and combinations thereof.

In an aspect, the amine carboxylic acid may be selected from the group consisting of glycine, β-alanine, 4-aminobutyric acid, 5-aminovaleric acid, 6-aminocaproic acid, 7-aminoheptanoic acid, 8-aminocaprylic acid, aminocaprylic acid, 12-aminolauric Acid, 14-aminotetradecanoic acid, 18-aminostearic acid, lysine, alanine, aspartic acid, glutamic acid, serine, threonine, alanine, valine, leucine, isoleucine, phenylalanine, tyrosine and combination thereof. In a further aspect, the amine carboxylic acid may be selected from the group consisting of β-alanine, 6-aminocaproic acid, 8-aminocaprylic acid, lysine, and combinations thereof.

In an aspect, the amine carboxylic acid may be selected from the group consisting of 3-aminopropanoic acid, 2-aminobutanoic acid, glycine, alanine, isoleucine, leucine, methionine, phenylalanine, valine, tryptophan, asparagine, glutamine, lysine, glutamic acid, and combinations thereof. In a further aspect, the amine carboxylic acid may be selected from the group consisting of 2-aminobutanoic acid, lysine, glutamic acid, and combinations thereof.

iii. Ratio

In an aspect, the molar ratio of the epoxy functional group from the epoxidized molecule to the carboxylic acid functional group (—COOH), the carboxylic anhydride functional group (—COOCO—), the hydroxyl functional group (—OH), or the amine functional group (—NH₂) of the crosslinker ranges from 2:1 to 1:2. In some aspect, the molar ratio of the epoxy functional group from the epoxidized molecule to the carboxylic acid functional group (—COOH), the carboxylic anhydride functional group (—COOCO—), the hydroxyl functional group (—OH), or the amine functional group (—NH₂) is about 2:1, about 1.5:1, about 1:1, about 1:1.5, or about 1:2.

In an aspect, the molar ratio of the epoxy functional group in the at least one epoxidized molecule to carboxylic acid functional group (—COOH) in the carboxylic acid comprising the structure of formula (II) is about 1:2 to about 2:1 In some aspects, the molar ratio of the epoxy functional group in the at least one epoxidized molecule to carboxylic acid functional group (—COOH) in the carboxylic acid comprising the structure of formula (II) is about 1:2, about 1:1.9, about 1:1.8, about 1:1.7, about 1:1.6, about 1:1.5, about 1:1.4, about 1:1.3, about 1:1.2, about 1:1.1, about 1:1, about 1.1:1, about 1.2:1, about 1.3:1, about 1.4:1, about 1.5:1, about 1.6:1, about 1.7:1, about 1.8:1, about 1.9:1, or about 2:1. In a further aspect, the molar ratio of the epoxy functional group in the at least one epoxidized molecule to carboxylic acid functional group (—COOH) in the carboxylic acid comprising the structure of formula (II) is about 1:1.5 to about 1.5:1.

In an aspect, the molar ratio of the epoxy functional group in the at least one epoxidized molecule to the anhydride functional group (—COOCO—) in the anhydride comprising the structure of formula (III) is about 1:1 to about 4:1. In some aspects, the molar ratio of the epoxy functional group in the at least one epoxidized molecule to the anhydride functional group (—COOCO—) in the anhydride comprising the structure of formula (III) is about 1:1, about 1.1:1, about 1.15:1, about 1.2:1, about 1.25:1, about 1.3:1, about 1.35:1, about 1.4:1, about 1.45:1, about 1.5:1, about 1.55:1, about 1.6:1, about 1.65:1, about 1.7:1, about 1.75:1, about 1.8:1, about 1.85:1, about 1.9:1, about 1.95:1, about 2:1, about 2.1:1, about 2.2:1, about 2.3:1, about 2.4:1, about 2.5:1, about 2.6:1, about 2.7:1, about 2.8:1, about 2.9:1, about 3:1, about 3.1:1, about 3.2:1, about 3.3:1, about 3.4:1, about 3.5:1, about 3.6:1, about 3.7:1, about 3.8:1, about 3.9:1, or about 4:1. In a further aspect, the molar ratio of the epoxy functional group in the at least one epoxidized molecule to the anhydride functional group (—COOCO—) in the anhydride comprising the structure of formula (III) is about 1.33:1 to about 3:1.

In an aspect, the molar ratio of the epoxy functional group in the at least one epoxidized molecule to the amine functional group (—NH₂) in the amine comprising the structure of formula (IV) is about 1:2 to about 2:1. In some aspects, the molar ratio of the epoxy functional group in the at least one epoxidized molecule to the amine functional group (—NH₂) in the amine comprising the structure of formula (IV) is about 1:2, about 1.1.9, about 1:1.8, about 1:1.7, about 1.1.6, about 1:1.5, about 1:1.4, about 1.1.3, about 1:1.2, about 1:1.1, about 1:1, about 1.1:1, about 1.2:1, about 1.3:1, about 1.4:1, about 1.5:1, about 1.6:1, about 1.7:1, about 1.8:1, about 1.9:1, or about 2:1. Ina further aspect, the molar ratio of the epoxy functional group in the at least one epoxidized molecule to the amine functional group (—NH₂) in the amine comprising the structure of formula (IV) is about 1:1.5 to 1.5:1.

In an aspect, the molar ratio of the epoxy functional group in the at least one epoxidized molecule to the hydroxyl functional group (—OH) in the alcohol comprising the structure of formula (V) is about 1:2 to about 2:1. In some aspects, the epoxy functional group in the at least one epoxidized molecule to the hydroxyl functional group (—OH) in the alcohol comprising the structure of formula (V) is about 1:2, about 1:1.9, about 1:1.8, about 1:1.7, about 1:1.6, about 1:1.5, about 1:1.4, about 1:1.3, about 1:1.2, about 1:1.1, about 1:1, about 1.1:1, about 1.2:1, about 1.3:1, about 1.4:1, about 1.5:1, about 1.6:1, about 1.7:1, about 1.8:1, about 1.9:1, or about 2:1. In a further aspect, the molar ratio of the epoxy functional group in the at least one epoxidized molecule to the hydroxyl functional group (—OH) in the alcohol comprising the structure of formula (V) is about 1:1.5 to 1.5:1.

In an aspect, the molar ratio of the epoxy functional group in the at least one epoxidized molecule to the carboxylic acid functional group (—COOH) in the hydroxyl carboxylic acid comprising the structure of formula (VI) is about 1:1 to about 4:1. In some aspects, the molar ratio of the epoxy functional group in the at least one epoxidized molecule to the carboxylic acid functional group (—COOH) in the hydroxyl carboxylic acid comprising the structure of formula (VI) is about 1:1, about 1.1:1, about 1.15:1, about 1.2:1, about 1.25:1, about 1.3:1, about 1.35:1, about 1.4:1, about 1.45:1, about 1.5:1, about 1.55:1, about 1.6:1, about 1.65:1, about 1.7:1, about 1.75:1, about 1.8:1, about 1.85:1, about 1.9:1, about 1.95:1, about 2:1, about 2.1:1, about 2.2:1, about 2.3:1, about 2.4:1, about 2.5:1, about 2.6:1, about 2.7:1, about 2.8:1, about 2.9:1, about 3:1, about 3.1:1, about 3.2:1, about 3.3:1, about 3.4:1, about 3.5:1, about 3.6:1, about 3.7:1, about 3.8:1, about 3.9:1, or about 4:1. Ina further aspect, the molar ratio of the epoxy functional group in the at least one epoxidized molecule to the carboxylic acid functional group (—COOH) in the hydroxyl carboxylic acid comprising the structure of formula (VI) is about 1.33:1 to about 3:1.

In an aspect, the molar ratio of the epoxy functional group in the at least one epoxidized molecule to the carboxylic acid functional group (—COOH) in the amine carboxylic acid comprising the structure of formula (VII) is about 1:1 to about 4:1. In some aspects, the molar ratio of the epoxy functional group in the at least one epoxidized molecule to the carboxylic acid functional group (—COOH) in the amine carboxylic acid comprising the structure of formula (VII) is about 1:1, about 1.1:1, about 1.15:1, about 1.2:1, about 1.25:1, about 1.3:1, about 1.35:1, about 1.4:1, about 1.45:1, about 1.5:1, about 1.55:1, about 1.6:1, about 1.65:1, about 1.7:1, about 1.75:1, about 1.8:1, about 1.85:1, about 1.9:1, about 1.95:1, about 2:1, about 2.1:1, about 2.2:1, about 2.3:1, about 2.4:1, about 2.5:1, about 2.6:1, about 2.7:1, about 2.8:1, about 2.9:1, about 3:1, about 3.1:1, about 3.2:1, about 3.3:1, about 3.4:1, about 3.5:1, about 3.6:1, about 3.7:1, about 3.8:1, about 3.9:1, or about 4:1. In a further aspect, the molar ratio of the epoxy functional group in the at least one epoxidized molecule to the carboxylic acid functional group (—COOH) in the amine carboxylic acid comprising the structure of formula (VII) is about 1.33:1 to about 3:1.

### B. Gel or Paste

In an aspect, the elastomer may be crumbled to form a crosslinked polyester elastomer powder.

In an aspect, a composition may be prepared by shearing the elastomer, or elastomer with a solvent, as described herein, to form a sheared gel. In another aspect, a composition may be prepared by combining the elastomer, as described herein, with a solvent thereby forming a mixture and shearing the mixture. In an aspect, the composition is a gel or a paste. In a further aspect, the composition is a gel.

In some aspects, the solvent will be as discussed below.

In some aspects, the viscosity of the gel may be from about 10 cp to about 1,000,000 cp as measured by rheometer at a shear rate of 0.1 s⁻¹. In another aspect, the viscosity of the gel may be from about 30,000 cp to about 500,000 cp. In some aspects, the viscosity of the gel may be about 10 cp, about 1,000 cp, about 5,000 cp, about 10,000 cp, about 15,000 cp, about 20,000 cp, about 25,000 cp, about 30,000 cp, about 35,000 cp, about 40,000 cp, about 45,000 cp, about 50,000 cp, about 55,000 cp, about 60,000 cp, about 65,000 cp, about 70,000 cp, about 75,000 cp, about 80,000 cp, about 85,000 cp, about 90,000 cp, about 95,000 cp, about 100,000 cp, about 150,000 cp, about 200,000 cp, about 250,000 cp, about 300,000 cp, about 350,000 cp, about 400,000 cp, about 450,000 cp, about 500,000 cp, about 550,000 cp, about 600,000 cp, about 650,000 cp, about 700,000 cp, about 750,000 cp, about 800,000 cp, about 850,000 cp, about 900,000 cp, about 950,000 cp, or about 1,000,000 cp.

In some aspects, the gel is comprised of particles of size from about 1 μm to about 500 μm as measured by laser diffraction particle size analyzer. In another aspect, the gel is comprised of particles of size from about 25 μm to about 400 μm. In some aspects, the gel is comprised of particles of size of about 1 μm, about 5 μm, about 10 μm, about 15 μm, about 20 μm, about 25 μm, about 30 μm, about 35 μm, about 40 μm, about 45 μm, about 50 μm, about 75 μm, about 100 μm, about 125 μm, about 150 μm, about 175 μm, about 200 μm, about 225 μm, about 250 μm, about 275 μm, about 300 μm, about 325 μm, about 350 μm, about 375 μm, or about 400 μm.

In an aspect, the elastomer may be prepared using the methods described herein.

### C. Methods

The disclosure also relates to a method of preparing an elastomer comprising reacting at least oe epoxidized molecule (e.g., epoxidized soybean oil) and at least one crosslinker (C₃₆ dimer acid and/or C₃₆ dimer diol) in the presence of a first solvent thereby forming a crosslinking polymer structure.

### D. Polymerization Reaction

In some aspects, the method of preparing the gel, as described herein, is produced in an environmentally friendly process. In additional aspects, the method of preparing the gel, as described herein, utilizes no toxic raw materials. In further aspects, the method of preparing the gel, as described herein, generates no toxic side products.

a. Components

i. Epoxidized Molecule

In an aspect, the at least one epoxidized molecule is a compound of formula (I) described above. In an aspect, the at least one epoxidized molecule may be an epoxidized vegetable oil described above, e.g., epoxidized soybean oil.

ii. Crosslinker

In an aspect, the at least one crosslinker is selected from the group consisting of:

(i) a carboxylic acid comprising the structure of formula (II) described above;

(ii) an anhydride comprising the structure of formula (III) described above;

(iii) an amine comprising the structure of formula (IV) described above;

(iv) an alcohol comprising the structure of formula (V) described above;

(v) a hydroxyl carboxylic acid comprising the structure of formula (VI) described above;

(vi) an amine carboxylic acid comprising the structure of formula (VII) described above.

In an aspect, the carboxylic acid comprises the structure of formula (II), wherein R² is C₂-C₆₀ alkyl group, C₂-C₆₀ heteroalkyl group, C₂-C₆₀ alkene group, or C₂-C₆₀ heteroalkene group; and n is an integer from 2 to 10. In a further aspect, R² may be succinyl, adipoyl, sebacoyl, dilinoleyl, or trilinoleyl.

In an aspect, the carboxylic acid comprises the structure of formula (II), wherein n is 2, 3, 4, 5, 6, 7, 8, 9, or 10. In an aspect, the carboxylic acid comprises the structure of formula (II), wherein n is an integer of 2 to 6. In an aspect, the carboxylic acid comprises the structure of formula (II), wherein n is 2, 3, 4, 5, or 6.

In an aspect, the carboxylic acid may be selected from the group consisting of citric acid, isocitric acid, aconitic acid, propane-1,2,3-tricarboxylic acid, trimesic acid, carballylic acid, C₅₄ trimer acid, mellitic acid, reaction product of ricinoleic acid and sebacic acid, reaction product of C₃₆ dimer acid and C₃₆ dimer diol, and combinations thereof. In a further aspect, the carboxylic acid may be selected from the group consisting of citric acid, C₅₄ trimer acid, and combinations thereof.

In an aspect, the carboxylic acid is a dicarboxylic acid. In an aspect, the dicarboxylic acid may be selected from the group consisting of malonic acid, succinic acid, glutaric acid, adipic acid, pimelic acid, suberic acid, azelaic acid, sebacic acid, undecanedioic acid, dodecanedioic acid, tridecanedioic acid, hexadecanedioic acid, C₂₁ dimer acid, C₃₆ dimer acid, hydrogenated C₃₆ dimer acid, aspartic acid, glutamic acid, tartaric acid, malic acid, and combinations thereof. In a further aspect, the dicarboxylic acid may be selected from the group consisting of malonic acid, succinic acid, adipic acid, azelaic acid, sebacic acid, undecanedioic acid, dodecanedioic acid, C₂₁ dimer acid, C₃₆ dimer acid, hydrogenated C₃₆ dimer acid, and combinations thereof. In another aspect, the dicarboxylic acid is selected from the group consisting of succinic acid, adipic acid, azelaic acid, sebacic acid, undecanedioic acid, dodecanedioic acid, C₂₁ dimer acid, C₃₆ dimer acid, hydrogenated C₃₆ dimer acid, and combinations thereof. In one aspect, the carboxylic acid is C₃₆ dimer acid.

In an aspect, C₃₆ dimer acid is the dicarboxylic acids prepared by dimerizing unsaturated fatty acids from plant oil.

In an aspect, C₅₄ trimer acid is the polycarboxylic acid prepared by trimerizing unsaturated fatty acids from plant oil.

In some aspects, the unsaturated fatty acids are palmitoleic acid, oleic acid, elaidic acid, vaccenic acid, linoleic acid, linoelaidic acid, or linolenic acid.

In some aspects, the plant oils are soybean oil, safflower oil, linseed oil, corn oil, sunflower oil, olive oil, canola oil, sesame oil, cottonseed oil, palm oil, rapeseed oil, tung oil, peanut oil, or milkweed oil.

In an aspect, the anhydride comprises the structure of formula (III), wherein R⁴ is C₂-C₁₀ alkyl group or C₂-C₁₀ alkene group. In a further aspect, R⁴ is C₂-C₈ alkyl group.

In an aspect, the anhydride may be selected from the group consisting of malonic anhydride, succinic anhydride, maleic anhydride, phthalic anhydride, glutaric anhydride, adipic anhydride, pimelic anhydride, suberic anhydride, azelaic anhydride, sebacic anhydride, undecanedioic anhydride, dodecanedioic anhydride, tridecanedioic anhydride, hexadecanedioic anhydride, C₂₁ dimer acid anhydride, C₃₆ dimer acid anhydride, hydrogenated C₃₆ dimer acid anhydride, aspartic anhydride, glutamic anhydride, tartaric anhydride, malic anhydride, and combinations thereof. In a further aspect, the anhydride may be selected from the group consisting of succinic anhydride, adipic anhydride, sebacic anhydride, C₃₆ dimer acid anhydride, hydrogenated C₃₆ dimer acid anhydride, and combinations thereof.

In an aspect, the C₃₆ dimer diamine is the diamine produced from a C₃₆ dimer acid.

In an aspect, the amine comprises the structure of formula (IV), wherein p is 2, 3, 4, 5, 6, 7, 8, 9, or 10. In an aspect, the amine comprises the structure of formula (IV), wherein p is an integer between 2 and 6. In an aspect, the amine comprises the structure of formula (IV), wherein p is 2, 3, 4, 5, or 6.

In an aspect, the amine may be selected from the group consisting of 1,2-diaminoethane, propane-1,3-diamine, butane-1,4-diamine, pentane-1,5-diamine, hexane-1,6-diamine, 1,7-heptanediamine, C₃₆ dimer diamine, hydrogenated C₃₆ dimer diamine, C₅₄ trimer triamine, and combinations thereof. In a further aspect, the amine may be selected from the group consisting of hexane-1,6-diamine. C₃₆ dimer diamine, hydrogenated C₃₆ dimer diamine, and combinations thereof.

In an aspect, the amine may be selected from the group consisting of include ethylenediamine, 1,3-diaminopropane (1,3-propanediamine), putrescine (1,4-butanediamine), cadaverine (1,5-pentanediamine), hexamethylenediamine (1,6-hexanediamine), trimethyl-1,6-hexanediamine, 1,7-heptanediamine, 1,8-octanediamine, 1,9-nonanediamine, 1,10-decanediamine, 1,11-undecanediamine, 1,12-dodecanediamine, diphenylethylenediamine, 1,2-diaminopropane, 1,2-diaminocyclohexane, triethylenetetramine, xylylenediamine, phenylenediamine, spermidine, spermine, polyethylenimine, C₃₆ dimer diamine, hydrogenated C₃₆ dimer diamine, poly(ethylene glycol) bis(amine), lysine, and combinations thereof. In some aspects, the amine may be selected from the group consisting of spermidine, spermine, polyethylenimine, and combinations thereof. In other aspects, the amine may be selected from the group consisting of 1,5-pentanediamine, 1,6-hexanediamine, 1,8-octanediamine, 1,10-decanediamine, 1,12-dodecanediamine, C₃₆ dimer diamine, hydrogenated C₃₆ dimer diamine, and combinations thereof.

In an aspect, wherein the amine may be a diamine. In an aspect, the diamine may be selected from the group consisting of ethylenediamine, 1,3-diaminopropane (1,3-propanediamine), putrescine (1,4-butanediamine), cadaverine (1,5-pentanediamine), hexamethylenediamine (1,6-hexanediamine), trimethyl-1,6-hexanediamine, 1,7-heptanediamine, 1,8-octanediamine, 1,9-nonanediamine, 1,10-decanediamine, 1,11-undecanediamine, 1,12-dodecanediamine, diphenylethylenediamine, 1,2-diaminopropane, 1,2-diaminocyclohexane, xylylenediamine, phenylenediamine, polyethylenimine, C₃₆ dimer diamine, hydrogenated C₃₆ dimer diamine, poly(ethylene glycol) bis(amine), lysine, and combinations thereof. In a further aspect, the diamine may be selected from the group consisting of 1,5-pentanediamine, 1,6-hexanediamine, 1,8-octanediamine, 1,10-decanediamine, 1,12-dodecanediamine, C₃₆ dimer diamine, hydrogenated C₃₆ dimer diamine, and combinations thereof.

In an aspect, the alcohol comprises the structure of formula (V), wherein R⁶ is C₂-C₂₀₀ alkyl group, C₂-C₂₀₀ heteroalkyl group, C₂-C₂₀₀ alkene group, or C₂-C₂₀₀ heteroalkene group; and q is an integer from 2 to 10. In a further aspect, the alcohol comprises the structure of formula (V), wherein R⁶ is C₂-C₆₀ alkyl group, C₂-C₆₀ heteroalkyl group, C₂-C₆₀ alkene group, or C₂-C₆₀ heteroalkene group; and q is an integer from 2 to 10.

In an aspect, the alcohol comprises the structure of formula (V), wherein q is 2, 3, 4, 5, 6, 7, 8, 9, or 10. In an aspect, the alcohol comprises the structure of formula (V), wherein q is an integer from 2 to 6. In an aspect, the alcohol comprises the structure of formula (V), wherein q is 2, 3, 4, 5, or 6.

In an aspect, the alcohol may be selected from the group consisting of glycerol, diglycerol, polyglycerol, sorbitan, castor oil, hydrogenated castor oil, sugar alcohol, monosaccharide, disaccharides, oligosaccharide, polysaccharides, tannin, gallic acid, gluconic acid, lactobionic acid, gluconolactone, and combinations thereof. In a further aspect, the alcohol may be selected from the group consisting of glycerol, diglycerol, polyglycerol, castor oil, hydrogenated castor oil, sorbitol, gallic acid, and combinations thereof. In another aspect, the alcohol may be selected from the group consisting of glycerol, diglycerol, polyglycerol, castor oil, hydrogenated castor oil, sorbitol, and combinations thereof.

In an aspect, the alcohol may be a diol. In some aspects, the diol may be selected from the group consisting of ethyleneglycol, 1,2-propanediol, 1,3-propanediol, 1,3-butanediol, 1,4-butanediol, 1,2-pentanediol, 1,3-pentanediol, 1,4-pentanediol 1,1,5-pentanediol, 1,2-hexanediol, 1,5-hexanediol, 1,6-hexanediol, C₃₆ dimer diol, hydrogenated C₃₆ dimer diol, and combinations thereof. In a further aspect, the diol may be selected from the group consisting of 1,3-propanediol, 1,3-butanediol, 1,4-butanediol, 1,5-pentanediol, 1,6-hexanediol, C₃₆ dimer diol, hydrogenated C₃₆ dimer diol, and combinations thereof. In another aspect, the diol may be selected from the group consisting of 1,3-propanediol, 1,3-butanediol, 1,4-butanediol, 1,5-pentanediol, 1,6-hexanediol, C₃₆ dimer diol, and combinations thereof. In one aspect, the alcohol is C₃₆ dimer diol. In an aspect, C₃₆ dimer diol is a diol produced from C₃₆ dimer acid.

In an aspect, the hydroxyl carboxylic acid may be selected from the group consisting of glycolic acid, lactic acid, salicylic acid, tropic acid, ricinoleic acid, isoricinoleic acid, lesquerolic acid, densipolic acid, auricolic acid, dimorphecolic acid, hydroxy palmitoleic acid, hydroxy palmitic acid, hydroxy oleic acid, 2-hydroxy stearic acid, 12-hydroxy stearic acid, and combinations thereof. In a further aspect, the hydroxyl carboxylic acid may be selected from the group consisting of ricinoleic acid, 12-hydroxy stearic acid, and combinations thereof.

In an aspect, the amine carboxylic acid may be selected from the group consisting of glycine, β-alanine, 4-aminobutyric acid, 5-aminovaleric acid, 6-aminocaproic acid, 7-aminoheptanoic acid, 8-aminocaprylic acid, aminocaprylic acid, 12-aminolauric Acid, 14-aminotetradecanoic acid, 18-aminostearic acid, lysine, alanine, aspartic acid, glutamic acid, serine, threonine, alanine, valine, leucine, isoleucine, phenylalanine, tyrosine and combination thereof. In a further aspect, the amine carboxylic acid may be selected from the group consisting of β-alanine, 6-aminocaproic acid, 8-aminocaprylic acid, lysine, and combinations thereof.

In an aspect, the amine carboxylic acid may be selected from the group consisting of 3-aminopropanoic acid, 2-aminobutanoic acid, glycine, alanine, isoleucine, leucine, methionine, phenylalanine, valine, tryptophan, asparagine, glutamine, lysine, glutamic acid, and combinations thereof. In a further aspect, the amine carboxylic acid may be selected from the group consisting of 2-aminobutanoic acid, lysine, glutamic acid, and combinations thereof.

iii. Ratio

In an aspect, the molar ratio of the epoxy functional group from the epoxidized molecule to the carboxylic acid functional group (—COOH), the carboxylic anhydride functional group (—COOCO—), the hydroxyl functional group (—OH), or the amine functional group (—NH₂) of the crosslinker is described above.

In an aspect, the molar ratio of the epoxy functional group in the at least one epoxidized molecule to carboxylic acid functional group (—COOH) in the carboxylic acid comprises the structure of formula (II) is described above.

In an aspect, the molar ratio of the epoxy functional group in the at least one epoxidized molecule to the anhydride functional group (—COOCO—) in the anhydride comprises the structure of formula (III) is described above.

In an aspect, the molar ratio of the epoxy functional group in the at least one epoxidized molecule to the amine functional group (—NH₂) in the amine comprises the structure of formula (IV) is described above.

In an aspect, the molar ratio of the epoxy functional group in the at least one epoxidized molecule to the hydroxyl functional group (—OH) in the alcohol comprises the structure of formula (V) is described above.

In an aspect, the molar ratio of the epoxy functional group in the at least one epoxidized molecule to the carboxylic acid functional group (—COOH) in the hydroxyl carboxylic acid comprises the structure of formula (VI) is described above.

In an aspect, the molar ratio of the epoxy functional group in the at least one epoxidized molecule to the carboxylic acid functional group (—COOH) in the amine carboxylic acid comprises the structure of formula (VII) is described above.

b. Catalyst

In an aspect, the reaction may further occur in the presence of at least one catalyst. In some aspects, the at least one catalyst may be selected from the group consisting of methanesulfonic acid, p-toluenesulfonic acid, benzene sulfonic acid, sulfuric acid, amidosulfonic acid, sulfamic acid, sodium bisulfate, phosphoric acid, hydrochloric acid, hydrobromic acid, nitric acid, sodium hydroxide, potassium hydroxide, sodium methoxide, sodium ethoxide, potassium methoxide, potassium ethoxide, bismuth neodecanoate, bismuth(III) citrate, bismuth(III) chloride, bismuth(III) acetate, bismuth(III) phosphate, tin chloride, tin-pyrone, dibutyltin dilaurate, di-nbutyl-oxo-stannane, butyl stannoic acid, zinc chloride, zinc bromide, zinc carboxylic salt, zinc oxide, zinc hydroxy nitrate salt, zinc hydroxy acetate, triethylamine, tripropylamine, cocamidopropyl dimethylamine, stearamidopropyl dimethylamine, isostearamidopropyl dimethylamine, and combinations thereof. In a further aspect, the at least one catalyst is p-toluenesulfonic acid, methanesulfonic acid, phosphoric acid, bismuth neodecanoate, or combinations thereof.

c. Solvent

In some aspects, the solvent may be biobased or naturally derived. In an aspect, the first solvent may be a triglyceride solvent, a mono-ester solvent, a di-ester solvent, a citrate ester solvent, an ether solvent, a carbonate solvent, a hydrocarbon solvent, a silicone solvent, and combinations thereof.

In an aspect, the solvent may be a triglyceride. In an aspect, the triglyceride solvent may be a compound of formula (VIII)

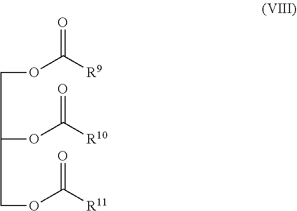

wherein

R⁹, R¹⁰, and R¹¹ are independently C₁-C₃₅ alkyl group, C₁-C₃₅ heteroalkyl group, C₂-C₃₅ alkene group, and C₂-C₃₅ heteroalkene group.

In an aspect, the compound of formula (VIII), wherein R⁹, R¹⁰, and R¹¹ are independently C₅-C₁₇ alkyl group or C₅-C₁₇ alkene group.

In an aspect, the triglyceride solvent may be selected from the group consisting of caprylic/capric triglyceride, triheptanoin, corn oil, soybean oil, olive oil, rape seed oil, cotton seed oil, coconut oil, almond oil, argon oil, rosehip oil, black seed oil, grape seed oil, avocado oil, apricot kernel oil, geranium oil, lavender oil, rosehip oil, macadamia oil, eucalyptus oil, sardine oil, herring oil, safflower oil, linseed oil, sunflower oil, olive oil, canola oil, sesame oil, cottonseed oil, palm oil, rapeseed oil, tung oil, fish oil, peanut oil, cuphea oil, milkweed oil, salicornia oil, whale oil, castor oil, and combinations thereof. In a further aspect, the triglyceride solvent may be selected from the group consisting of caprylic/capric triglyceride, triheptanoin, and combinations thereof.

In an aspect, the solvent may be a mono-ester. In an aspect, the mono-ester solvent may be a compound of formula (IX)

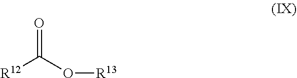

wherein

R¹² is C₁-C₃₅ alkyl group, C₁-C₃₅ heteroalkyl group, C₂-C₃₅ alkene group, or C₂-C₃₅ heteroalkene group; and

R¹³ is H, C₁-C₃₅ alkyl group, C₁-C₃₅ heteroalkyl group, C₂-C₃₅ alkene group, or C₂-C₃₅ heteroalkene group.

In an aspect, the compound of formula (IX), wherein R¹² is C₅-C₁₇ alkyl group or C₅-C₁₇ alkene group and R¹³ is C₂-C₁₈ alkyl group.

In an aspect, the mono-ester solvent may be selected from the group consisting of coco-caprylate/caprate, coco-caprylate, coco-caprate, jojoba oil, jojoba esters, isopropyl jojobate, ethyl macadamiate, isoamyl laurate, heptyl undecylenate, methylheptyl isostearate, isostearyl isostearate, glyceryl ricinoleate, isostearyl palmitate, myristyl myristate, octyldodecyl myristate, octyldodecyl hydroxystearate, butyl myristate, ethylhexyl cocoate, ethylhexyl palmitate, ethylhexyl stearate, butyl stearate, decyl oleate, isocetyl behenate, isocetyl myristate, isocetyl palmitate, isocetyl stearate, isodecyl oleate, isopropyl isostearate, isopropyl myristate, isopropyl palmitate, oleyl oleate, propylene glycol laurate, octydodecyl erucate, C₁₂-C₁₃ alkyl lactate, C₁₂-C₁₅ alkyl lactate, isostearyl lactate, glycereth-5 lactate, lauryl lactate, myristyl lactate, oleyl lactate, laureth-2 benzoate, C₁₂-C₁₅ alkyl benzoate, C₁₂-C₁₅ pareth-3 benzoate, dipropylene glycol benzoate, isodecyl salicylate, C₁₂-C₁₅ alkyl salicylate, tridecyl salicylate, ethylhexyl isononanoate, cetyl ethylhexanoate, isononyl isononanoate, isodecyl ethylhexanoate, isodecyl isononanoate, tridecyl ethylhexanoate, isotridecyl isononanoate, isostearyl isononanoate, cetearyl isononanoate, laureth-2 ethylhexanoate, cetearyl ethylhexanoate, isodecyl neopentanoate, isostearyl neopentanoate, nyristyl neopentanoate, isostearyl behenate, octyldodecyl neopentanoate, tridecyl neopentanoate, and combinations thereof.

In an aspect, the mono-ester solvent may be selected from the group consisting of coco-caprylate/caprate, coco-caprylate, jojoba oil, isoamyl laurate, methylheptyl isostearate, C₁₂-C₁₃ alkyl lactate, C₁₂-C₁₅ alkyl lactate, ethylhexyl isononanoate, cetyl ethylhexanoate, isononyl isononanoate, isodecyl ethylhexanoate, isodecyl isononanoate, tridecyl ethylhexanoate, isotridecyl isononanoate, isostearyl isononanoate, cetearyl isononanoate, and combinations thereof. In a further aspect, the mono-ester solvent may be selected from the group consisting of coco-caprylate/caprate, coco-caprylate, isoamyl laurate, isononyl isononanoate, heptyl undecylenate, jojoba oil, jojoba esters, and combinations thereof. In one aspect, the mono-ester solvent is coco-caprylate/caprate.

In an aspect, the solvent may be a di-ester solvent. In an aspect, the di-ester solvent may be a compound of formula (X), formula (XI), or formula (XII)

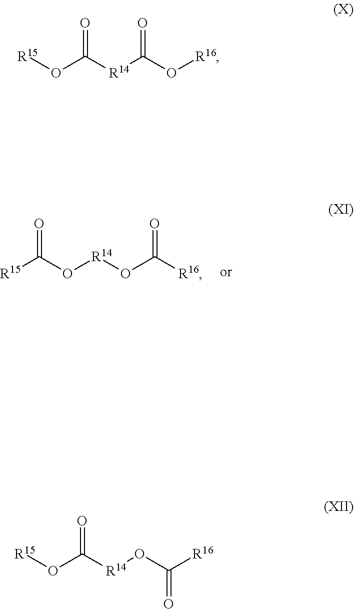

wherein

R¹⁴ is C₁-C₃₅ alkyl group, C₁-C₃₅ heteroalkyl group, C₂-C₃₅ alkene group, or C₂-C₃₅ heteroalkene group; and\

R¹⁵ and R¹⁶ are independently H, C₁-C₃₅ alkyl group, C₁-C₃₅ heteroalkyl group, C₂-C₃₅ alkene group, or C₂-C₃₅ heteroalkene group.

In an aspect, the compound of formula (X), formula (XI), or formula (XII), wherein R¹⁴ is C₂-C₁₀ alkyl group or C₂-C₁₀ alkene group and R¹⁵ and R¹⁶ are independently C₁-C₁₂ alkyl group or C₂-C₁₂ alkene group.

In an aspect, the di-ester solvent may be selected from the group consisting of diethyl succinate, dibutyl succinate, diethyhexyl succinate, diisopropyl sebacate, dimethyl sebacate, diethyl sebacate, dibutyl sebacate, diisostearyl dimer, diisostearyl malate, isostearyl stearoyl stearate, isocetyl stearoyl stearate, octyldodecyl stearoyl stearate, diethylhexyl malate, diethylhexyl maleate, dipropylene glycol dibenzoate, dicapryl adipate, dicaprylyl maleate, diisopropyl dimer, diisopropyl adipate, diisobutyl adipate, diisopropyl sebacate, diisostearyl dimer, diethyhexyl succinate, diethylene glycol diethylhexanoate, neopentyl glycol dicaprate, propylene glycol dicaprylate/dicaprate, neopentyl glycol diisostearate, neopentyl glycol diethylhexanoate, neopentyl glycol diheptanoate, and combinations thereof. In a further aspect, the di-ester solvent may be selected from the group consisting of dicapryl adipate, dicaprylyl maleate, diisopropyl adipate, diisobutyl adipate, diethyl succinate, dibutyl succinate, diethyhexyl succinate, diisopropyl sebacate, dimethyl sebacate, diethyl sebacate, dibutyl sebacate, neopentyl glycol diethylhexanoate, neopentyl glycol diheptanoate, and combinations thereof.

In an aspect, the solvent may be a citrate ester. In an aspect, the citrate ester may be a compound of formula (XIII)

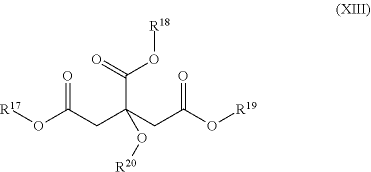

wherein

R¹⁷, R¹⁸, R¹⁹, and R²⁰ are independently H, C₁-C₃₅ alkyl group, C₁-C₃₅ heteroalkyl group, C₂-C₃₅ alkene group, or C₂-C₃₅ heteroalkene group.

In an aspect, the compound of formula (XIII), wherein R¹⁷, R¹⁸, and R¹⁹ are independently C₁-C₁₀ alkyl group or C₂-C₁₀ alkene group and R²⁰ is an acetyl group.

In an aspect, the citrate ester solvent may be a compound selected from the group consisting of tricaprylyl citrate, triisostearyl citrate, triisocetyl citrate, trioctyldodecyl citrate, triethyl citrate, tributyl citrate, acetyl triethyl citrate, acetyl tributyl citrate, trioctyldodecyl citrate, triisocetyl citrate, and combinations thereof.

In an aspect, the solvent may be an ether solvent. In an aspect, the ether solvent may be a compound of formula (XIV)

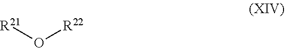

wherein

R²¹ and R²² are independently H, C₂-C₂₀ alkyl group, C₂-C₂₀ heteroalkyl group, C₂-C₂₀ alkene group, or C₂-C₂₀ heteroalkene group.

In an aspect, the compound of formula (XIV), wherein R^{2′} and R²² are independently C₂-C₂₀ alkyl group.

In an aspect, the ether solvent may be selected from the group consisting of dicaprylyl ether, didecyl ether, panthenyl ethyl ether, dicetyl ether, dimyristyl ether, distearyl ether, distearyl ether, dilauryl ether, and combinations thereof. In a further aspect, the ether solvent may be selected from the group consisting of dicaprylyl ether, didecyl ether, and combinations thereof.

In an aspect, the solvent may be a carbonate solvent. In an aspect, the carbonate solvent may be a compound of formula (XV)

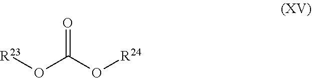

wherein

R²³ and R²⁴ are independently H, C₂-C₂₀ alkyl group, C₂-C₂₀ heteroalkyl group, C₂-C₂₀ alkene group, or C₂-C₂₀ heteroalkene group.

In an aspect, the compound of formula (XV), wherein R²³ and R²⁴ are independently C₂-C₂₀ alkyl group.

In an aspect, the carbonate solvent may be selected from the group consisting of dicaprylyl carbonate, diethylhexyl carbonate, and combinations thereof.

In an aspect, the hydrocarbon solvent is selected from the group consisting of farnesene, hydrogenated farnesene, coconut alkanes, coconut/palm kernel alkanes, C₉-C₁₂ alkane, C₁₀-C₁₃ alkane, alkane, C₁₂-C₁₄ alkane, C₁₃-C₁₅ alkane, alkane, C₁₄-C₁₉ alkane, C₁₄-C₂₀ alkane, C₁₄-C₂₂ alkane, C₁₅-C₁₉ alkane, C₁₁-C₂₈ alkane, C₁₇-C₂₃ alkane, C₉-C₁₂ isoalkane, C₉-C₁₃ isoalkane, C₉-C₁₄ isoalkane, C₉-C₁₆ isoalkane, C₁₀-C₁₁ isoalkane, C₁₀-C₁₂ isoalkane, C₁₀-C₁₃ isoalkane, isoalkane, C₁₁-C₁₃ isoalkane, isoalkane, C₁₂-C₁₄ isoalkane, C₁₂-C₁₅ isoalkane, C₁₂-C₂₀ isoalkane, C₁₃-C₁₄ isoalkane, C₁₃-C₁₆ isoalkane, C₁₄-C₁₆ isoalkane, C₁₅-C₁₉ isoalkane, diethylhexylcyclohexane, undecane, tridecane, tetradecane, pentadecane, hexadecane, octadecane, docosane, squalane, hydrogenated polyisobutene, polybutene, hydrogenated polydecene, hydrogenated didecene, mineral oil, paraffin, isoparaffin, paraffium liquidum, petrolatum, dodecane, isohexadecane, isododecane, isoeicosane, and combinations thereof. In a further aspect, hydrocarbon solvent is selected from the group consisting of farnesene, hydrogenated farnesene, coconut alkanes, C₉-C₁₂ alkane, C₁₃-C₁₅ alkane, C₁₄-C₁₉ alkane, C₁₄-C₂₀ alkane, C₁₄-C₂₂ alkane, C₁₅-C₁₉ alkane, C₁₃-C₁₆ isoalkane, dodecane, undecane, tridecane, tetradecane, pentadecane, hexadecane, octadecane, squalane, isododecane, isohexadecane, and combinations thereof.

In an aspect, the solvent may be a silicone solvent. In some aspects, the silicone solvent may be selected from the group consisting of dimethicone, phenyl dimethicone, caprylyl methicone, ethyl trisiloxane, cyclotetrasiloxane, cyclopentasiloxane, cyclohexasiloxane, and combinations thereof.

In an aspect, the amount of the first solvent in the reaction may be present from about 10% to about 70% of the total weight of the epoxidized vegetable oil, the crosslinker, and the solvent. In an aspect, the amount of the first solvent in the reaction may be present from about 20% to about 70% of the total weight of the epoxidized vegetable oil, the crosslinker, and the solvent. In a further aspect, the amount of the solvent present may be from about 30% to about 50% of the total weight of the epoxidized vegetable oil, the crosslinker, and the solvent. In another aspect, the amount of the solvent present may be about 10%, about 15%, about 20%, about 25%, about 30%, about 35%, about 40%, about 45%, about 50%, about 55%, about 60%, about 65%, or about 70% of the total weight of the epoxidized vegetable oil, the crosslinker, and the solvent.

d. Temperature

In an aspect, the reaction can occur at a temperature from about 10° C. to about 250° C. In an aspect, the reaction may occur at a temperature from about 15° C. to about 250° C., from about 15° C. to about 225° C., from about 20° C. to about 220° C., from about 25° C. to about 200° C., from about 25° C. to about 175° C., from about 20° C. to about 175° C., or from about 20° C. to about 150° C. In a further aspect, the reaction can occur at a temperature from about 25° C. to about 200° C. In yet a further aspect, the reaction can occur at a temperature from about 20° C. to about 150° C. In another aspect, the reaction can occur at a temperature of about 10° C., about 15° C., about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., about 95° C., about 100° C., about 105° C., about 110° C., about 115° C., about 120° C., about 125° C., about 130° C., about 135° C., about 140° C., about 145° C., about 150° C., about 155° C., about 160° C., about 165° C., about 170° C., about 175° C., about 180° C., about 185° C., about 190° C., about 195° C., about 200° C., about 205° C., about 210° C., about 215° C., about 220° C., about 225° C., about 230° C., about 235° C., about 240° C., about 245° C., or about 250° C. In one aspect, the reaction can occur at a temperature from about 70° C. to about 140° C.

e. Time

In an aspect, the reaction time can be from about 4 hours to about 100 hours. In some aspects, the reaction time can be from about 4 hours to about 80 hours, from about 4 hours to about 50 hours, from about 4 hours to about 25 hours, from about 8 hours to about 25 hours, or from about 8 hours to about 24 hours. In another aspect, the reaction time can be about 4, about 10, about 15, about 20, about 25, about 30, about 35, about 40, about 45, about 50, about 55, about 60, about 65, about 70, about 75, about 80, about 85, about 90, about 95, or about 100 hours. In yet another aspect, the reaction time can be about 8, about 10, about 12, about 14, about 16, about 18, about 20, about 22, or about 24 hours. In one aspect, the reaction time is about 12 hours to about 80 hours.

f. Swelling the Crosslinking Polymer Structure

In an additional aspect, the method can further comprise (i) combining the crosslinking polymer structure with a second solvent thereby forming a swollen crosslinking polymer structure and (ii) subjecting the swollen crosslinking polymer structure to shear force thereby forming a uniform polyester elastomer which can be processed into a gel. The second solvent can be one or more of the first solvents listed above.

In an aspect, the amount of the second solvent in the method may be from about 100 wt. % to about 900 wt. % of the crosslinking polymer. In another aspect, the amount of the second solvent in the method may be about 100 wt. %, about 150 wt. %, about 200 wt. %, about 250 wt. %, about 300 wt. %, about 350 wt. %, about 400 wt. %, about 450 wt. %, about 500 wt. %, about 550 wt. %, about 600 wt. %, about 650 wt. %, about 700 wt. %, about 750 wt. %, about 800 wt. %, about 850 wt. %, or about 900 wt. % of the crosslinking polymer. In an aspect, the amount of the second solvent can be dependent on the crosslinking polymer structure.

i. Shear Force

In some aspects, the shear force may be provided by any type of mixing and shearing equipment. In a further aspect, the mixing and shearing equipment may be batch mixer, planetary mixer, single or multiple screw extruder, dynamic or static mixer, colloid mill, homogenizer, sonolator, or a combination thereof.

g. Properties

The viscosity of the gel prepared from an elastomer described herein is described above. Additionally, the gel is comprised of particles of size described above.

### E. Personal Care Formulations

In an additional aspect of the present disclosure, the products of the present disclosure, i.e., the gel prepared from a thermoset elastomer, may be formulated into a personal care formulation.

In some aspects, the personal care formulation may be a cosmetic or a medical product.

In an aspect, the personal care formulation further comprises a preservative, an antioxidant, a chelating agent, a gum or thickener, an oil, a wax, a fragrance, an essential oil, an emulsifier, a surfactant, and combinations thereof.

In some aspects, the personal care formulation may be formulated with a preservative, an antioxidant, a chelating agent, a gum or thickener, an oil, a wax, a fragrance, an essential oil, an emulsifier, a surfactant, and combinations thereof.

In some aspects, the products of the present disclosure may be added to formulations comprising make-ups, color cosmetics, foundations, blushes, lipsticks, lip balms, eyeliners, mascaras, oil removers, color cosmetic removers, and powders

In some aspects, the products of the present disclosure may function as delivery systems for oil and water soluble substances such as vitamins.

In one aspect, the products of the present disclosure are compatible with a particulate additive. In one aspect, the particulate additive is an inorganic particulate, polymeric latex, and/or a pigment. In another aspect, the polymers are capable of suspending these particles for a prolonged period in personal care formulations.

Once the desired emulsion is prepared, the resulting material is usually a high viscosity cream with good feel characteristics and high absorbance of volatile solvents. The emulsion can then be blended into personal care formulations for hair care, skin care, and the like.

The personal care formulation can be a personal care application including deodorants, antiperspirants, antiperspirant/deodorants, shaving products, skin lotions, moisturizers, toners, bath products, cleansing products, hair care products such as shampoos, conditioners, mousses, styling gels, hair sprays, hair dyes, hair color products, hair bleaches, waving products, hair straighteners, manicure products (e.g., nail polish, nail polish remover, nail creams and lotions, cuticle softeners), protective creams (e.g., sunscreen, insect repellent and anti-aging products), color cosmetics (e.g., lipsticks, foundations, face powders, eye liners, eye shadows, blushes, makeup, and mascaras). The personal care application can also be a drug delivery system for topical application of a medicinal composition that can be applied to the skin.

In one aspect, the personal care formulation further comprises one or more personal care ingredients. Suitable personal care ingredients include, without limit, emollients, moisturizers, humectants, pigments (e.g., pearlescent pigments such as bismuth oxychloride and titanium dioxide coated mica), colorants, fragrances, biocides, preservatives, antioxidants, anti-fungal agents, antiperspirant agents, exfoliants, hormones, enzymes, medicinal compounds, vitamins, salts, electrolytes, alcohols, polyols, absorbing agents for ultraviolet radiation, botanical extracts, surfactants, silicone oils, organic oils, waxes, film formers, thickening agents (e.g., fumed silica or hydrated silica), particulate fillers (e.g., talc, kaolin, starch, modified starch, mica, nylon, clays, such as, for example, bentonite and organo-modified clays).

In some aspects, the one or more personal care components included in the personal care formulations are selected from the group consisting of a humectant, emollient, moisturizer, pigment, colorant, fragrance, biocide, preservative, antioxidant, anti-fungal agent, antiperspirant agent, exfoliant, hormone, enzyme, medicinal compound, vitamin, salt, electrolyte, alcohol, polyol, absorbing agent for ultraviolet radiation, botanical extract, surfactant, silicone oil, organic oil, wax, film former, and thickening agent. In some aspects, the one or more emollients is selected from the group consisting of triglyceride esters, wax esters, alkyl or alkenyl ester of fatty acids, polyhydric alcohol esters, and mixtures thereof. In some aspects, the one or more personal care components is a silicone oil, an organic oil, or mixtures thereof.

In one aspect, the personal care formulation is an antiperspirant composition that comprises a polymer composition or product described herein and one or more active antiperspirant agents. Suitable antiperspirant agents include, but are not limited to, the Category I active antiperspirant ingredients listed in the U.S. Food and Drug Administration's Oct. 10, 1993 Monograph on antiperspirant drug products for over-the-counter human use including aluminum halides, aluminum hydroxyhalides, for example, aluminum chlorohydrate, and complexes or mixtures thereof with zirconyl oxyhalides and zirconyl hydroxyhalides, (e.g., aluminum-zirconium chlorohydrate, and aluminum zirconium glycine complexes, such as aluminum zirconium tetrachlorohydrex gly).

In another aspect, the personal care formulation is a skin care composition comprising a polymer composition or product described herein, and a vehicle, such as a silicone oil or an organic oil. The skin care composition can also include emollients, such as triglyceride esters, wax esters, alkyl or alkenyl esters of fatty acids or polyhydric alcohol esters, pigments, vitamins (e.g., Vitamin A, Vitamin C and Vitamin E), sunscreen or sunblock compounds (e.g., titanium dioxide, zinc oxide, oxybenzone, octylmethoxy cinnamate, butylmethoxy dibenzoylm ethane, p-aminobenzoic acid and octyl dimethyl-p-aminobenzoic acid).

In yet another aspect, the personal care formulation is a color cosmetic composition such as a lipstick, a makeup or mascara. The color cosmetic composition comprises a polymer composition or product described herein and a coloring agent (e.g., pigment, water-soluble dye, or liposoluble dye).

In still yet another aspect, the personal care formulation comprises a thermoset elastomer described herein and fragrant materials. The fragrant materials can be fragrant compounds, encapsulated fragrant compounds or fragrance releasing compounds that either the neat compounds or are encapsulated.

### F. Use of the Gels

In one aspect, the disclosure relates to the use of a gel composition described herein for personal care formulation, such as a personal care application. The personal care applications can be any described above.

### EXAMPLES

The following examples are included to demonstrate various aspects of the present disclosure. It should be appreciated by those of skill in the art that the techniques disclosed in the examples that follow represent techniques discovered by the inventors to function well in the practice of the disclosure, and thus can be considered to constitute preferred modes for its practice. However, those of skill in the art should, in light of the present disclosure, appreciate that many changes can be made in the specific examples which are disclosed and still obtain a like or similar result without departing from the spirit and scope of the disclosure.

### Example 1: Polymerization from Epoxidized Soybean Oil and Dicarboxylic Acid to Prepare Polymer Elastomer

In a suitable vessel equipped with agitation, heat and an ability to distill off water, 100 g of epoxidized soybean oil was added along with 90 g of hydrogenated dimer acid. Next 190 g of hemisqualane was added as solvent. After all ingredients have been charged under agitation, the temperature of the mass was raised to 140° C., and water was stripped off if needed. The temperature was held for 12-20 hours or until gelation took, place and polymer elastomer was formed. Thereafter the elastomer was broken into a powder by mechanical stirring.

### Example 2: Polymerization from Epoxidized Soybean Oil and Carboxylic Anhydride to Prepare Polymer Elastomer

In a suitable vessel equipped with agitation, heat and an ability to distill off water, 100 g of epoxidized soybean oil was added along with 16 g of succinic anhydride. Next 58 g of dicaprylyl ether was added as solvent. After all ingredients have been charged under agitation, the temperature of the mass was raised to 140° C., and water was stripped off if needed. The temperature was held for 12-20 hours or until gelation took, place and polymer elastomer was formed. Thereafter the elastomer was broken into a powder by mechanical stirring.

### Example 3: Polymerization from Epoxidized Soybean Oil and Diamine to Prepare Polymer Elastomer

In a suitable vessel equipped with agitation, heat and an ability to distill off water, 110 g of epoxidized soybean oil was added along with 30 g of 1,10-diaminodecane. Next 140 g hemisqualane was added as solvent. After all ingredients have been charged under agitation, the temperature of the mass was raised to 140° C., and water was stripped off if needed. The temperature was held for 12-20 hours or until gelation took, place and polymer elastomer was formed. Thereafter the elastomer was broken into a powder by mechanical stirring.

### Example 4: Polymerization from Epoxidized Soybean Oil, Hydroxylstearic Acid and Diacid to Prepare Polymer Elastomer

In a suitable vessel equipped with agitation, heat and an ability to distill off water, 95 g of epoxidized soybean oil was added along with 30 g of sebacic acid and 9 g of 12-hydroxylstearic acid. Next 130 g of dicaprylyl ether was added as solvent. After all ingredients have been charged under agitation, the temperature of the mass was raised to 180° C., and water was stripped off if needed. The temperature was held for 10-15 hours or until gelation took place and polymer elastomer was formed. Thereafter the elastomer was broken into a powder by mechanical stirring.

### Example 5: Preparation of Polymer Gel from Polymer Elastomer

After a very nice elastomer was produced which did not separate upon standing, 350 g of MCT oil was added into the vessel to mix with 120 g of elastomer. After the gel and solvent mixture sit in vessel for 12 hours it was homogenized with an Gaulin homogenizer to produce a creamy, translucent gel of very smooth consistency, suitable for use in personal care formulations.

### Example 6: Preparation of a Natural Oil/Water Foundation

Components of a natural oil/water foundation are listed in Table 1.

| | INCI name | % |
|---|---|---|
| A | Aqua | Up to 100.0 |
| | Xanthan gum | 3.0 |
| | Glycerin | 3.0 |
| | Disodium EDTA | 0.05 |
| | Hydroxyethyl Acrylate/Sodium | 2.0 |
| | Acryloyldimethyl Taurate Copolymer & | |
| | Squalane & Polysorbate 60 | |
| B | PEG-20 Methyl Glucose Sesquistearate | 3.5 |
| | Methyl Glucose Sesquistearate | 2.0 |
| | Cl 77891 (and) Triethoxycaprylylsilane | 5.03 |
| | Cl 77492 (and) Triethoxycaprylylsilane | 0.67 |
| | Cl 77491 (and) Triethoxycaprylylsilane | 0.204 |
| | Cl 77499 (and) Triethoxycaprylylsilane | 0.096 |
| | Cetyl Alcohol | 1.5 |
| | C13-15 Alkane | 4.0 |
| | Ethylhexyl Stearate | 3.0 |
| | Coco-Caprylate/Caprate | 2.0 |
| | Isononyl Isononanoate | 4.0 |
| | Gel from example 5 | 5.0 |
| C | Preservative | q.s. |
| | Fragrance | q.s. |

Combine phase A ingredients and heat to 50° C. with stirring. Wet powders with solvent mixture in phase B and homogenize. Add the homogenized mixture to the rest of phase B and heat to 50° C. Add phases B into phase A and mix for 10 minutes until content is homogeneous. Add phase C and homogenize.

### Example 7: Preparation of a Natural Water/Oil Foundation

| | INCI name | % |
|---|---|---|
| A | Water | Up to 100 |
| | Glycerin | 3.0 |
| | Sodium Chloride | 1.0 |
| B | PEG-30 Dipolyhydroxystearate | 5.0 |
| | Polyglyceryl-3 Diisostearate | 2.0 |
| | Pentaerythrityl Distearate | 1.0 |
| | Dicaprylyl Carbonate (and) Stearalkonium | 6.0 |
| | Hectorite (and) Propylene Carbonate | |
| | Isononyl Isononanoate | 4.0 |
| | Coco-Caprylate | 4.0 |
| | Propylheptyl Caprylate | 7.0 |
| | Gel from example 5 | 5.0 |
| | CI 77891 (and) Triethoxycaprylylsilane | 4.19 |
| | CI 77492 (and) Triethoxycaprylylsilane | 0.56 |
| | CI 77491 (and) Triethoxycaprylylsilane | 0.17 |
| | CI 77499 (and) Triethoxycaprylylsilane | 0.08 |
| | Hordeum Vulgare Seed Flour | 2.0 |
| | Aluminum Starch Octenylsuccinate | 4.0 |
| | Silica | 1.0 |
| | Boron Nitride | 1.0 |
| C | Phenoxyethanol (and) Ethylhexylglycerin | 1.0 |

Mix phase A and heat to 50° C. Wet powders with solvents and homogenize until smooth. Add to the rest of phase B and heat to 50° C. Slowly add phase A to phase B while mixing. Homogenize phase A+B. Cool down and add phase® C.

### Example 8: Preparation of a Matte Lipstick with Pigments

| Phase | INCI name | % |
|---|---|---|
| A | Ricinus Communis (Castor) Seed Oil | To 100 |
| | Titanium Dioxide (CI 77891)) | 2.00 |
| | CI 77499 | 0.04 |
| | CI 77492 | 0.40 |
| | CI 45410 | 0.50 |
| | Helianthus Annuus (Sunflower) Seed Oil | 18.00 |
| | Euphorbia Cerifera (Candelilla) Wax | 15.00 |
| | Butyrospermum Parkii (Shea) Butter | 5.00 |
| | Cocos Nucifera | 20.00 |
| | Gel from example 5 | 25.00 |
| | Neossance Hemisqualane | 5.00 |
| | Caprylyl Caprylate/Caprate | 20.00 |

Combine the phase (A) ingredients and heat to 70 C until homogenous. Pour into mold.

### Example 9: Preparation of a Natural Primer

| Phase | INCI Name | Wt (%) |
|---|---|---|
| A | Gel from example 5 | 70.0 |
| | Caprylic/Capric Triglyceride (and) Polyurethane-79 | 5.0 |
| | Heptyl Undecylenate | 25 |

Mix all ingredients and heat to 80-90° C. using cowles mixer. Ensure the oil thickener is dissolved, then pour the mixture into the jar and let it cool overnight.

### Example 10: Preparation of a Moisturizing Skin Cream

| Phase | INCI Name | Wt (%) |
|---|---|---|
| A | Aqua | Up to 100.0 |
| | Xanthan Gum | 0.2 |
| | Glycerin | 0.5 |
| B | Disodium EDTA | 0.05 |
| | Glyceryl Stearate Citrate | 3.0 |
| | Sodium Stearoyl Glutamate | 0.5 |
| | Glyceryl Stearate | 1.5 |
| | Cetearyl Alcohol | 2.0 |
| | C13-15 Alkane | 5.0 |
| | PPG-15 Stearyl Ether | 5.0 |
| | Caprylic-Capric Triglyceride | 5.0 |
| | Gel from example 5 | 5.0 |
| C | Preservative | q.s. |
| | Fragrance | q.s. |

Mix all the ingredients of phase A, heat to 75° C. Mix all the ingredients of phase B, heat to 70° C. Add phase B to phase A with intensive stirring. Homogenize the mixture for 1-2 minutes. Cool to 50° C. under gentle stirring. Add preservative and fragrance as desired, and mix well.

### Example 11: Preparation of an Oil in Water Sunscreen

| Phase | INCI Name | Wt (%) |
|---|---|---|
| A | Water | Up to 100.0 |
| | Xanthan gum | 0.2 |
| | Glycerin | 0.5 |
| | Disodium EDTA | 0.1 |
| | Carbomer | 0.2 |
| | Butylene glycol | 2.0 |
| B | Sodium stearoyl glutamate | 1.0 |
| | Glyceryl stearate | 3.0 |
| | Ethylhexyl salicylate | 5.0 |
| | Homosalate | 15.0 |
| | Avobenzone | 3.0 |
| | Octocrylene | 10 |
| C | Gel from example 5 | 5.0 |
| D | Preservative | as needed |

Wet xanthan gum with glycerin and butylene glycol; add the rest of the ingredients of phase A and heat to 40° C. until homogenous. Mix ingredients of phase B and heat to 70-80° C. until homogenous. Add phase C to B and mix. Slowly add phase B+C to A. Homogenize the mixture 11,000 rpm for 1 minute. Cool down and add preservative.

### OTHER ASPECTS

All publications, patents, and patent applications mentioned in this specification are incorporated herein by reference in their entirety to the same extent as if each individual publication, patent, or patent application was specifically and individually indicated to be incorporated by reference in its entirety. Where a term in the present application is found to be defined differently in a document incorporated herein by reference, the definition provided herein is to serve as the definition for the term.

While the present disclosure has been described in connection with specific aspects thereof, it will be understood that present disclosure is capable of further modifications and this application is intended to cover any variations, uses, or adaptations of the invention following, in general, the principles of the invention and including such departures from the present disclosure that come within known or customary practice within the art to which the invention pertains and can be applied to the essential features hereinbefore set forth, and follows in the scope of the claimed.

What is claimed is:

1. An elastomer comprising the reaction product of at least one epoxidized molecule and at least one crosslinker.

2. The elastomer of claim 1, wherein the at least one epoxidized molecule is a compound of formula (I) 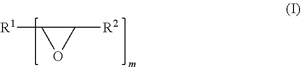
wherein
R¹ is C₆-C_{100,000} alkyl group, C₆-C_{100,000} heteroalkyl group, C₆-C_{100,000} alkene group, C₆-C_{100,000} heteroalkene group, C₆-C_{100,000} alkyne group, C₆-C_{100,000} heteroalkyne group, C₆-C_{100,000} cyclic group, or C₆-C_{100,000} heterocyclic group;
R² is hydrogen, C₁-C_{100,000} alkyl group, C₁-C_{100,000} heteroalkyl group, C₂-C_{100,000} alkene group, C₂-C_{100,000} heteroalkene group, C₂-C_{100,000} alkyne group, C₂-C_{100,000} heteroalkyne group, C₃-C_{100,000} cyclic group, or C₃-C_{100,000} heterocyclic group; and
m is an integer from 2 to 1,000.

3. The elastomer of claim 1, wherein the at least one epoxidized molecule is at least one epoxidized vegetable oil.

4. The elastomer of claim 1, wherein the at least one crosslinker is selected from the group consisting of:
(i) a carboxylic acid comprising the structure of formula (II) 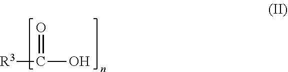
wherein
R³ is C₂-C₂₀₀ alkyl group, C₂-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group; and
n is an integer from 2 to 10;
(ii) an anhydride comprising the structure of formula (III) 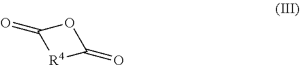
wherein
R⁴ is C₂-C₂₀₀ alkyl group, C₂-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group;
(iii) an amine comprising the structure of formula (IV) 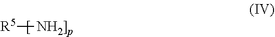
wherein
R⁵ is C₂-C₂₀₀ alkyl group, C₂-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group; and
p is an integer from 2 to 10;
(iv) an alcohol comprising the structure of formula (V) 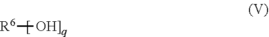
wherein
R⁶ is C₂-C₂₀₀ alkyl group, C₂-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group;
q is an integer from 2 to 10;
(v) a hydroxyl carboxylic acid comprising the structure of formula (VI) 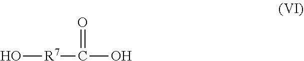
wherein
R⁷ is C₁-C₂₀₀ alkyl group, C₁-C₂₀₀ heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group;
(vi) a amine carboxylic acid comprising the structure of formula (VII) 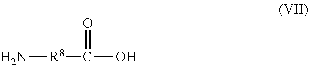
wherein
R⁸ is C₁-C₂₀₀ alkyl group, C₁-C_{100,000} heteroalkyl group, C₂-C₂₀₀ alkene group, C₂-C₂₀₀ heteroalkene group, C₂-C₂₀₀ alkyne group, C₂-C₂₀₀ heteroalkyne group, C₃-C₂₀₀ cyclic group, or C₂-C₂₀₀ heterocyclic group;
and combinations thereof.

5. The elastomer of claim 4, wherein the at least one crosslinker is a carboxylic acid comprising the structure of formula (II) wherein R² is C₂-C₆₀ alkyl group, C₂-C₆₀ heteroalkyl group, C₂-C₆₀ alkene group, or C₂-C₆₀ heteroalkene group; and n is an integer from 2 to 6.

6. The elastomer of claim 4, wherein the carboxylic acid is selected from the group consisting of citric acid, isocitric acid, aconitic acid, propane-1,2,3-tricarboxylic acid, trimesic acid, carballylic acid, C₅₄ trimer acid, mellitic acid, reaction product of ricinoleic acid and sebacic acid, reaction product of C₃₆ dimer acid and C₃₆ dimer diol, and combinations thereof.

7. The elastomer of claim 5, wherein the carboxylic acid is a dicarboxylic acid.

8. The elastomer of claim 7, wherein the dicarboxylic acid is selected from the group consisting of malonic acid, succinic acid, glutaric acid, adipic acid, pimelic acid, suberic acid, azelaic acid, sebacic acid, undecanedioic acid, dodecanedioic acid, tridecanedioic acid, hexadecanedioic acid, C₂₁ dimer acid, C₃₆ dimer acid, hydrogenated C₃₆ dimer acid, aspartic acid, glutamic acid, tartaric acid, malic acid, and combinations thereof. In a further aspect, the dicarboxylic acid may be selected from the group consisting of malonic acid, succinic acid, adipic acid, azelaic acid, sebacic acid, undecanedioic acid, dodecanedioic acid, C₂₁ dimer acid, C₃₆ dimer acid, hydrogenated C₃₆ dimer acid, and combinations thereof.

9. The elastomer of claim 5, wherein the at least one crosslinker is an alcohol comprising the structure of formula (V) wherein R⁶ is C₂-C₂₀₀ alkyl group, C₂-C₂₀₀ heteroalkyl group, C₂-C₂₀₀ alkene group, or C₂-C₂₀₀ heteroalkene group; and q is an integer from 2 to 6.

10. The elastomer of claim 9, wherein the alcohol is a diol.

11. The elastomer of claim 10, wherein the diol is selected from the group consisting of ethyleneglycol, 1,2-propanediol, 1,3-propanediol, 1,3-butanediol, 1,4-butanediol, 1,2-pentanediol, 1,3-pentanediol, 1,4-pentanediol, 1,5-pentanediol, 1,2-hexanediol, 1,5-hexanediol, 1,6-hexanediol, C₃₆ dimer diol, hydrogenated C₃₆ dimer diol, and combinations thereof.

12. The elastomer of claim 1 prepared by reacting at least one epoxidized molecule and at least one crosslinker in the presence of a first solvent thereby forming a crosslinking polymer structure.

13. The elastomer of claim 12, wherein the solvent is selected from the group consisting of a triglyceride solvent, a mono-ester solvent, a di-ester solvent, a citrate ester solvent, an ether solvent, a carbonate solvent, a hydrocarbon solvent, a silicone solvent, and combinations thereof.

14. The elastomer of claim 13, wherein the triglyceride solvent comprising the compound of formula (VIII) 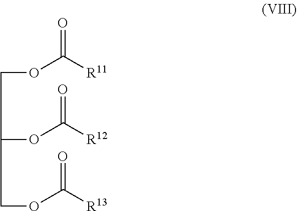
wherein
R¹¹, R¹², and Rⁿ are independently C₁-C₃₅ alkyl group, C₁-C₃₅ heteroalkyl group, C₂-C₃₅ alkene group, or C₂-C₃₅ heteroalkene group.

15. The elastomer of claim 13, wherein the triglyceride solvent is selected from the group consisting of caprylic/capric triglyceride, triheptanoin, corn oil, soybean oil, olive oil, rape seed oil, cotton seed oil, coconut oil, almond oil, argon oil, rosehip oil, black seed oil, grape seed oil, avocado oil, apricot kernel oil, geranium oil, lavender oil, rosehip oil, macadamia oil, eucalyptus oil, sardine oil, herring oil, safflower oil, linseed oil, sunflower oil, olive oil, canola oil, sesame oil, cottonseed oil, palm oil, rapeseed oil, tung oil, fish oil, peanut oil, cuphea oil, milkweed oil, salicornia oil, whale oil, castor oil, and combinations thereof.

16. The elastomer of claim 13, wherein the mono-ester solvent is a compound of formula (IX) 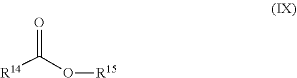
wherein
R¹⁴ is C₁-C₃₅ alkyl group, C₁-C₃₅ heteroalkyl group, C₂-C₃₅ alkene group, or C₂-C₃₅ heteroalkene group; and
R¹⁵ is H, C₁-C₃₅ alkyl group, C₁-C₃₅ heteroalkyl group, C₂-C₃₅ alkene group, or C₂-C₃₅ heteroalkene group.

17. The elastomer of claim 13, wherein the mono-ester solvent is selected from the group consisting of coco-caprylate/caprate, coco-caprylate, coco-caprate, jojoba oil, jojoba esters, isopropyl jojobate, ethyl macadamiate, isoamyl laurate, heptyl undecylenate, methylheptyl isostearate, isostearyl isostearate, glyceryl ricinoleate, isostearyl palmitate, myristyl myristate, octyldodecyl myristate, octyldodecyl hydroxystearate, butyl myristate, ethylhexyl cocoate, ethylhexyl palmitate, ethylhexyl stearate, butyl stearate, decyl oleate, isocetyl behenate, isocetyl myristate, isocetyl palmitate, isocetyl stearate, isodecyl oleate, isopropyl isostearate, isopropyl myristate, isopropyl palmitate, oleyl oleate, propylene glycol laurate, octydodecyl erucate, Cu-CD alkyl lactate, C₁₂-C₁₅ alkyl lactate, isostearyl lactate, glycereth-5 lactate, lauryl lactate, myristyl lactate, oleyl lactate, laureth-2 benzoate, C₁₂-C₁₅ alkyl benzoate, C₁₂-C₁₅ pareth-3 benzoate, dipropylene glycol benzoate, isodecyl salicylate, C₁₂-C₁₅ alkyl salicylate, tridecyl salicylate, ethylhexyl isononanoate, cetyl ethylhexanoate, isononyl isononanoate, isodecyl ethylhexanoate, isodecyl isononanoate, tridecyl ethylhexanoate, isotridecyl isononanoate, isostearyl isononanoate, cetearyl isononanoate, laureth-2 ethylhexanoate, cetearyl ethylhexanoate, isodecyl neopentanoate, isostearyl neopentanoate, nyristyl neopentanoate, isostearyl behenate, octyldodecyl neopentanoate, tridecyl neopentanoate, and combinations thereof.

18. The elastomer of claim 13, wherein the di-ester solvent is a compound of formula (X), formula (XI), or formula (XII) 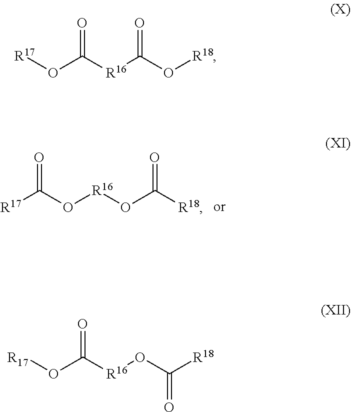
wherein
R¹⁶ is C₁-C₃₅ alkyl group, C₁-C₃₅ heteroalkyl group, C₂-C₃₅ alkene group, or C₂-C₃₅ heteroalkene group; and
R¹⁷ and V are independently H, C₁-C₃₅ alkyl group, C₁-C₃₅ heteroalkyl group, C₂-C₃₅ alkene group, or C₂-C₃₅ heteroalkene group.

19. The elastomer of claim 13, wherein the citrate ester is a compound of formula (XIII) 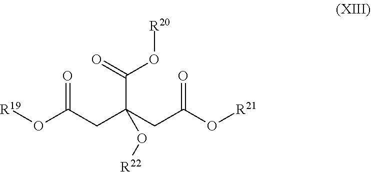
wherein
R¹⁹, R²⁰, R²¹, and R²² are independently H, C₁-C₃₅ alkyl group, C₁-C₃₅ heteroalkyl group, C₂-C₃₅ alkene group, or C₂-C₃₅ heteroalkene group.

20. The elastomer of claim 13, wherein the ether solvent is a compound of formula (XIV) 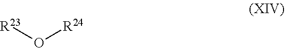
wherein
R²³ and R²⁴ are independently H, C₂-C₂₀ alkyl group, C₂-C₂₀ heteroalkyl group, C₂-C₂₀ alkene group, or C₂-C₂₀ heteroalkene group.

21. The elastomer of claim 13, wherein the ether solvent is selected from the group consisting of dicaprylyl ether, didecyl ether, panthenyl ethyl ether, dicetyl ether, dimyristyl ether, distearyl ether, distearyl ether, dilauryl ether, and combinations thereof.

22. The elastomer of claim 13, wherein the carbonate solvent is a compound of formula (XV) 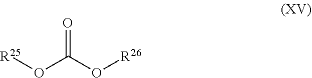
wherein
R²⁵ and R²⁶ are independently H, C₂-C₂₀ alkyl group, C₂-C₂₀ heteroalkyl group, C₂-C₂₀ alkene group, or C₂-C₂₀ heteroalkene group.

23. The elastomer of claim 13, wherein the hydrocarbon solvent is selected from the group consisting of farnesene, hydrogenated farnesene, coconut alkanes, coconut/palm kernel alkanes, C₉-C₁₂ alkane, C₁₀-C₁₃ alkane, alkane, C₁₂-C₁₇ alkane, C₁₃-C₁₅ alkane, C₁₄-C₁₇ alkane, C₁₄-C₁₉ alkane, C₁₄-C₂₀ alkane, C₁₄-C₂₂ alkane, C₁₅-C₁₉ alkane, C₂₁-C₂₈ alkane, C₁₇-C₂₃ alkane, C₉-C₁₂ isoalkane, C₉-C₁₃ isoalkane, C₉-C₁₄ isoalkane, C₉-C₁₆ isoalkane, C₁₀-C₁₁ isoalkane, C₁₀-C₁₂ isoalkane, C₁₀-C₁₃ isoalkane, isoalkane, C₁₁-C₁₃ isoalkane, C₁₁-C₁₄ isoalkane, C₁₂-C₁₄ isoalkane, Cu-Cis isoalkane, C₁₂-C₂₀ isoalkane, C₁₃-C₁₄ isoalkane, C₁₃-C₁₆ isoalkane, C₁₄-C₁₆ isoalkane, C₁₅-C₁₉ isoalkane, diethylhexylcyclohexane, undecane, tridecane, tetradecane, pentadecane, hexadecane, octadecane, docosane, squalane, hydrogenated polyisobutene, polybutene, hydrogenated polydecene, hydrogenated didecene, mineral oil, liquidum, petrolatum, dodecane, isohexadecane, isododecane, isoeicosane, and combinations thereof.

24. A composition comprising the elastomer of claim 1.

25. The composition of claim 24, wherein the composition is a gel.

26. A gel produced by combining an elastomer of claim 1 with one or more solvents and processed by homogenization.

27. A personal care formulation comprising the gel of claim 26.

28. The personal care formulation of claim 27, wherein the personal care formulation is a personal care application selected from the group consisting of a deodorant, an antiperspirant, a skin cream, a facial cream, a hair shampoo, a hair conditioner, a mousse, a hair styling gel, a hair spray, a protective cream, a lipstick, a facial foundations, blushes, makeup, and mascara, a skin care lotion, a moisturizer, a facial treatment, a personal cleanser, a facial cleanser, a bath oil, a perfume, a shaving cream, a pre-shave lotion, an after-shave lotion, a cologne, a sachet, and a sunscreen.

29. A method of preparing an elastomer of claim 1 comprising reacting at least one epoxidized molecule and at least one crosslinker in the presence of a first solvent thereby forming a crosslinking polymer structure.

30. The method of claim 29, wherein the method further comprises:
i. combining the crosslinking polymer structure in a second solvent thereby forming a swollen crosslinking polymer structure; and
ii. subjecting the swollen crosslinking polymer structure to shear force thereby forming the gel.
